(19) United States
(12) Patent Application Publication
(10) Pub. No.: US 2026/0283945 A1
(43) Pub. Date: Sep. 24, 2026

(54) Novel alginate and cellulose derivative based composition of the liquid anti-reflux formulation

(71) Applicants: NUTRITION & BIOSCIENCES USA 1, LLC, Wilmington, DE (US); DANISCO INDIA PVT LTD, New Delhi (IN)

(72) Inventors: Atul Ashok Kumar Lohade, New Delhi (IN); Vinay Muley, New Delhi (IN); Rathmaker Palarapu, New Delhi (IN); Matthias Knarr, Bomlitz (DE)

(21) Appl. No.: 19/100,628

(22) PCT Filed: Aug. 2, 2023
(86) PCT No.: PCT/US2023/071476

§ 371 Date: Feb. 3, 2025

(30) Foreign Application Priority Data
Aug. 2, 2022 (IN) 202211044288

(51) Int. Cl. A61K 9/00; A61K 31/717; A61K 31/734; A61K 33/10

(52) U.S. Cl. CPC A61K 9/0065; A61K 31/717; A61K 31/734; A61K 33/10

(57) ABSTRACT

The present invention relates to pharmaceutical raft-forming suspensions suitable for oral treating gastroesophageal reflux diseases, methods for its preparation, as well as the use in the treatment of a gastroesophageal reflux disease.

### FIELD OF THE INVENTION

The present invention relates to pharmaceutical raft-forming suspensions suitable for oral treating gastroesophageal reflux diseases, methods for its preparation, as well as the use in the treatment of a gastroesophageal reflux disease.

### BACKGROUND OF THE INVENTION

The human stomach is a complex system which has different functions, such as to store food, to initiate the digestion of proteins, to kill harmful bacteria and to move food into the small intestine as a pasty material called chyme.

When an individual consumes food, the swallowed food is passed through the oesophagus to the stomach by wavelike contractions known as peristalsis. The lumen of the terminal portion of the oesophagus is slightly narrowed because of a thickening of the circular muscle fibres in its wall: the lower oesophageal or gastro-oesophageal sphincter. After food passes into the stomach, constriction of the muscle fibre prevents the stomach contents from regurgitating into the oesophagus. Regurgitating would occur because of pressure differences on both sides as a result of respiratory movements.

Gastroesophageal reflux arises when the oesophageal sphincter opens spontaneously or fails to close for varying periods of time, causing the acidic gastric juices to rise, together with food, from the stomach to the oesophagus, and sometimes even to the mouth. Gastroesophageal reflux disease arises when the episodes of reflux take place at extended periods of time. The disease or syndrome is due to incomplete closure of the cardiac sphincter at the top of the stomach, and symptoms may range from a burning sensation to oesophagitis, nerve reflexes on the vagus nerve, thickening of the oesophageal area (hiatus hernia), and mutations of the epithelium (Barrett's oesophagus). Gastroesophageal reflux disease may also promote oesophageal cancer, or it may worsen or contribute to respiratory disorders (such as asthma, chronic cough, and pulmonary fibrosis). When a reflux of acids from the stomach comes into contact with the lining of the oesophagus, it not only causes direct damage to the mucosa, but can also cause a burning sensation in the chest and throat known as “heartburn”. The disease can give rise to serious complications, especially a chronic inflammatory state (reflux oesophagitis) which, with time, can cause bleeding and alterations of the mucosa that narrow the oesophageal lumen and can make swallowing difficult.

Alginate compositions described in GB1524740 form the basis of the product Gaviscon. In a strongly acid environment like the stomach, thick, neutral gels form which are able to float on the gastric juices and form a thick, dense layer called a “raft”, which acts as a plug for gastroesophageal reflux and inhibits acid regurgitation from the stomach to the oesophagus.

WO2012128520 relates to a liquid composition for treating a gastroesophageal disease by oral administration.

### SUMMARY OF THE INVENTION

It is an object of embodiments of the invention to provide pharmaceutical raft-forming suspensions suitable for the treatment of gastroesophageal reflux diseases.

The present invention relates in a broad aspect to raft-forming suspensions comprising alginate in a relatively small amount compared to traditional suspensions, which suspensions have comparable raft strength and are suitable for treatment of gastroesophageal reflux diseases.

Accordingly, in a first aspect the present invention relates to pharmaceutical raft-forming suspension suitable for the treatment by oral administration of gastroesophageal reflux diseases comprising or consisting of
a) an alginate in the amount of 2% (w/w) to 5% (w/w);
b) a cellulose derivative polymer selected from the group consisting of hydroxypropyl methylcellulose (HPMC), carboxymethyl cellulose (CMC), such as sodium carboxymethylcellulose, and methylcellulose (MC), hydroxypropyl cellulose (HPC); and mixtures thereof;
c) a carbonate, such as an alkali metal carbonate, such as sodium carbonate or sodium bicarbonate; or ammonium- or calcium carbonate;
d) a multivalent alginate cross-linking ion, such as ions of calcium, magnesium, aluminium, such as from calcium carbonate in an amount of about 1.1% (w/w) to 2.2% (w/w);
e) at least one viscosifier selected from i) a hydrocolloid, such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as a carbomer, such as Carbomer Type-A; and
f) water or other pharmaceutical vehicle.

It is to be understood that components c) and d) of the suspension compositions described herein, i.e. the carbonate and the multivalent alginate cross-linking ion respectively may be the same compound, such as wherein this carbonate source for the floating of the raft is e.g. calcium carbonate; which calcium carbonate also provides for the multivalent cross-linking ion.

In a second aspect the present invention relates to a method for the preparation of a raft-forming suspension comprising the steps of mixing the components of
a) an alginate in the amount of 2% (w/w) to 5% (w/w);
b) a cellulose derivative polymer selected from the group consisting of hydroxypropyl methylcellulose (HPMC), carboxymethyl cellulose (CMC), such as sodium carboxymethylcellulose, and methylcellulose (MC), hydroxypropyl cellulose (HPC); and mixtures thereof;
c) a carbonate, such as an alkali metal carbonate, such as sodium carbonate or sodium bicarbonate; or ammonium- or calcium carbonate;
d) a multivalent alginate cross-linking ion, such as ions of calcium, magnesium, aluminium, such as from calcium carbonate in an amount of about 1.1% (w/w) to 2.2% (w/w);
e) at least one viscosifier selected from i) a hydrocolloid, such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as a carbomer, such as Carbomer Type-A; and
f) water or other pharmaceutical vehicle.

In a third aspect the present invention relates to the use of a pharmaceutical raft-forming suspension comprising
a) an alginate in the amount of 2% (w/w) to 5% (w/w);
b) a cellulose derivative polymer selected from the group consisting of hydroxypropyl methylcellulose (HPMC), carboxymethyl cellulose (CMC), such as sodium carboxymethylcellulose, and methylcellulose (MC), hydroxypropyl cellulose (HPC); and mixtures thereof;
c) a carbonate, such as an alkali metal carbonate, such as sodium carbonate or sodium bicarbonate; or ammonium- or calcium carbonate;
d) a multivalent alginate cross-linking ion, such as ions of calcium, magnesium, aluminium, such as from calcium carbonate in an amount of about 1.1% (w/w) to 2.2% (w/w);
e) at least one viscosifier selected from i) a hydrocolloid, such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as a carbomer, such as Carbomer Type-A; and
f) water or other pharmaceutical vehicle; for use in the treatment of a gastroesophageal reflux disease.

### DETAILED DESCRIPTION OF THE INVENTION

### Alginic Acid and Salts Thereof.

Alginates, derived from, inter alia, brown seaweeds are linear, unbranched bio-polymers consisting of (1-4)-linked β-D-mannuronic acid (M) and α-L-guluronic acid (G) residues. Alginates are not random copolymers but consist of blocks of similar and alternating sequences of residues, for example, MMMM, GGGG, and GMGM. In extracted form alginate absorbs water quickly. The physical properties of alginates may depend on the relative proportion of the M and

G blocks. Gel formation at neutral pH requires a multivalent ion, such as calcium, magnesium, aluminium, such as from a calcium source to provide calcium ion to interact with G-blocks. The greater the proportion of these G-blocks, the greater the gel strength.

As used herein a “multivalent alginate cross-linking ion” refers to any ion suitable for cross-link or for gel-formation of alginate. It is to be understood that this alginate gel formation may be provided by many multivalent ions including bivalent alkaline earth cations (Mg2+, Ca2+, and Sr2+), bivalent transition metal ions (Mn2+, Co2+, Cu2+, and Zn2+) as well as trivalent metal cations (Fe3+, Cr3+, Al3+, Ga3+, Sc3+, and La3+). In some specific embodiments, multivalent alginate cross-linking ion used is calcium ions or magnesium ions or aluminium ions

“Alginate” is the term usually used for the salts of alginic acid, but it can also refer to all the derivatives of alginic acid and alginic acid itself; Alginate is present in the cell walls of brown algae as the calcium, magnesium and sodium salts of alginic acid. Dry, powdered, sodium alginate or potassium alginate may be obtained from an extraction process of this brown algae. The seaweed residue is then removed by filtration and the remaining alginate may then be recovered from the aqueous solution.

Another way to recover the alginate from the initial extraction solution is to add a calcium salt. This causes calcium alginate to form with a fibrous texture; it does not dissolve in water and can be separated from it. The separated calcium alginate is suspended in water and acid is added to convert it into alginic acid.

Alginates suitable for use in the practice of this invention will typically have a molecular weight such that they exhibit a viscosity in the range of 5-1,000 mPa·s. when measured at 2 wt % at 20° C. using rheometer setup with cup and bob geometry at a shear rate of 10 s⁻¹. In some embodiment, such alginates will exhibit a viscosity of between 6 and 600 mPa·s, such as between 7 and 500 mPa·s, or between 8 and 500 mPa·s when so measured. In some other embodiment, such alginates will exhibit a viscosity of between 8 and 400 mPa·s, such as between 8 and 300 mPa·s, such as between 9 and 200 mPa·s, or between 10 and 100 mPa·s when so measured.

In some embodiments according to the present invention, a high G type alginate is used. A high G type alginate means that the alginate(s) employed in the practice of the present invention possess an average of at least 50 percent adjacent G units. In some embodiments the alginate will possess an average of at least 52 percent adjacent G units; in other embodiments such alginate will possess an average of at least 55 percent or more of adjacent G units, in other embodiments such alginate will possess an average of at least 60, 65, or 70 percent or more of adjacent G units, as such higher the content of adjacent G units may result in improved product textures.

It has been found that a microdispersion of alginic acid containing a plasticizer in the correct ratios or amounts is capable of forming an alginic acid film. The alginic film shows controlled drug permeation over time in a Franz cell diffusion test. Only a very low film thickness (59 μm) is required for a firm, but flexible film providing controlled release, such as for controlled release of zero order. It is to be understood that within the context of the present invention a significantly lower alginate concentration is present as compared to existing alginate raft-forming suspensions: The composition provides desired raft characteristics with significantly reduced alginate quantity (40% w/w compared to the amount used in reference Gaviscon composition). Furthermore, there is a reduced raft strength variability: With reduced alginate and addition of sodium carboxymethylcellulose, the composition showed reduced raft strength variability (% RSD below 15%) compared to reference formulation (% RSD above 20%). This may provide consistency in raft strength performance in vivo.

According to the present invention, alginate, such as alginic acid or an alginate salt is present in the amount of 2% (w/w) to 5% (w/w) based on the total weight of the composition. In the present invention, alginate refers to any alginic acid or an alginate salt, such as sodium alginate, magnesium alginate, potassium alginate, triethanolamine alginate or propylene glycol monoglycolate.

### Viscosifiers

Viscosifiers or viscosifying agents are known to the person skilled in the art. Suitable viscosifiers include hydrocolloids such as any one selected from the group consisting of: guar gum, pectin and its derivatives, xanthan gum, arabinoxylan, cellulose and its derivatives, chitin, xylan, beta-glucan, gum Arabic, hyaluronic acid, and gelatin; and ii) a pharmaceutically acceptable poly-acrylic acid, such as a carbomer, such as Carbomer Type-A. For the purpose of the present invention, a viscosifyer does not include alginate or cellulose derivative polymers as specifically defined elsewhere.

### Cellulose Derivative Polymers

Any suitable cellulose derivative polymer may be used according to the present invention. The person skilled in the art will know these suitable polymers.

Suitable film forming polymer used according to the present invention includes low-viscosity hydroxypropylcellulose (HPC), ethylcellulose (EC), methylcellulose (MC), carboxymethyl cellulose (CMC), and hydroxypropyl methylcellulose (HPMC), such as hypromellose 2910 (7-12% HP, 28-30% methoxy), hypromellose 2906 (4-7.5% HP, 27-30% methoxy), Hypromellose 2208 (4-12% HP, 19-24% methoxy), Hypromellose 1828 (23-32% HP, 16.5-20% methoxy).

Commercially available carboxymethyl cellulose (CMC) include TEXTURACEL™ from IFF, Celetec™ from CPKelco, Aqualon™ from Ashland, Rheoflo® from USK Kimya A.S. and Akucell® from Nouryon (former AkzoNobel). Commercially available methylcelluloses and hydroxypropyl methylcelluloses include Japanese Pharmacopoeia METOLOSE (trademark) series and METOLOSE series for food additives from Shin-Etsu Chemical Co., Ltd., AnyCoat-C or AnyAddy (trademark) series from Lotte (former Samsung) Fine Chemicals Co., Ltd., METHOCEL (trademark) series from IFF (former DOW Chemical Company), and Benecel (trademark) series from Ashland.

The preservative may be any suitable compound known in the art of pharmaceutically acceptable compounds, such as any one selected from the group consisting of ethanol, benzethonium chloride, citric acid monohydrate, sodium salicylate, carbol, sodium benzoate, sodium dehydroacetate, oxyquinoline sulfate, potassium sorbate, benzalkonium chloride, benzeneconium chloride, Honey, 2-propanol, formalin, 1,2-hydroxypropane, human serum albumin, potassium L-glutamate, N-coconut oil fatty acid acyl-N-carboxymethyl-N-hydroxyethylethylenediamine sodium, thimerosal, boric acid, taurine, Sodium edetate, N-hexadecylviridinium chloride, 4-chloro-3-methylphenol, m-cresol, cresol, phenylethanol, 1,2-benzisothiazolin-3-one, disodium sulfite, glycerol (II) sulfate, phosphoric acid, butyl glycidyl ether, dl-camphor, sodium citrate, chlorobutanol, 2-hydroxybenzoic acid, phenyl salicylate, thymol, paraform, benzyl alcohol, sodium tetraborate, L-menthol, carboxybenzene, ethyl parahydroxybenzoate, butyl parahydroxybenzoate, propyl parahydroxybenzoate, methyl parahydroxybenzoate, methyl paraoxybenzoate, eucalyptus oil, chlorhexidine gluconate, butylhydroxytoluene, sorbic acid, borneol, β-naphthol, dehydroacetic acid, Isobutyl p-oxybenzoate, Peru balsam in color, benzoin, agar, 2-mercaptobenzimidazole, isopropyl p-oxybenzoate, n-dodecyltrimethylammonium chloride Tea Tree Oil, glyceryl caprate, polyglyceryl-2 laurate, polyglyceryl-10 laurate, ethylhexyl glycerin, glyceryl caprylate, or a combination of two or more of them.

Methylcellulose is one suitable cellulose derivative polymer to the present invention. Methylcellulose has anhydroglucose units joined by 1-4 linkages. Each anhydroglucose unit contains hydroxyl groups at the 2, 3, and 6 positions. Partial or complete substitution of these hydroxyls with methoxyl groups creates methylcellulose. For example, treatment of cellulosic fibers with caustic solution, followed by a methylating agent, yields cellulose ethers substituted with one or more methoxyl groups. If not further substituted with other alkyls, this cellulose ether is known as methylcellulose. Methylcellulose is characterized by the weight percent of methoxyl groups. By convention, the weight percent is an average weight percentage based on the total weight of the cellulose repeat unit, including all substituents. The content of the methoxyl group is reported based on the mass of the methoxyl group (i.e. —OCH3). The determination of the % methoxyl in methylcellulose (MC) polymer is carried out according to the United States Pharmacopeia (USP 37, “Methylcellulose”, pages 3776-3778). The % methoxyl can be converted into degree of substitution (DS) for methyl substituents, DS (methyl). DS (methyl), also designated as DS (methoxyl), of a methylcellulose is the average number of OH groups substituted with methyl groups per anhydroglucose unit. Preferably, methylcellulose has % methoxyl of 18% or more; more preferably 25% or more. Preferably, ingredient (b) has % methoxyl of 50% or less; more preferably 40% or less; and even more preferably 35% or less. Even more preferably, methylcellulose has a DS (methyl) of 1.55 or higher; more preferably 1.65 or higher; and most preferably 1.70 or higher. DS (methyl) is preferably 2.25 or lower; more preferably 2.20 or lower; and most preferably 2.10 or lower. In some embodiments according to the present invention a relevant characterization of methylcellulose is the quotient s23/s26. The numerals 2, 3, and 6 refer to the carbon atoms on the anyhdroglucose units, defined.

The parameter s23 is the molar fraction of anhydroglucose units wherein only the two hydroxy groups in the 2- and 3-positions of the anhydroglucose unit are substituted with methyl groups, and the parameter s26 is the molar fraction of anhydroglucose units wherein only the two hydroxy groups in the 2- and 6-positions of the anhydroglucose unit are substituted with methyl groups. For determining the s23, the term “the molar fraction of anhydroglucose units wherein only the two hydroxy groups in the 2- and 3-positions of the anhydroglucose unit are substituted with methyl groups” means that the two hydroxy groups in the 2- and 3-positions are substituted with methyl groups and the 6-positions are unsubstituted hydroxy groups. For determining the s26, the term “the molar fraction of anhydroglucose units wherein only the two hydroxy groups in the 2- and 6-positions of the anhydroglucose unit are substituted with methyl groups” means that the two hydroxy groups in the 2- and 6-positions are substituted with methyl groups and the 3-positions are unsubstituted hydroxy groups. The quotient s23/s26 is determined by dividing s23 by s26. According to the present invention in some embodiments the s23/s26 is 0.24 or less, such as 0.23 or less. Moreover, s23/s26 may be 0.10 or more, such as 0.14 or more. Methylcellulose having such s23/s26 ratio can be produced as generally described in International Patent Application, publication No. WO 2013/059064. A specific process for producing a methylcellulose having an above-mentioned s23/s26 ratio is described in WO2017192445 and commercial product with such s23/s26 ratio is Methocel™ Bind 112 from IFF.

The present invention also requires the raft-forming suspension to contain a carbonate source for the floating of the raft, such as an alkali metal carbonate, such as an alkali metal bicarbonate or an alkali metal carbonate, such as sodium carbonate or sodium bicarbonate, or ammonium carbonate, or ammonium bicarbonate, calcium carbonate, or any other carbonate or bicarbonate salt. It is also required that the raft-forming suspension contains a multivalent ion for alginate cross-linking, such as calcium, magnesium, aluminium, such as from calcium carbonate in an amount of about 1.1% (w/w) to 2.2% (w/w).

The solution for the viscosity measurements of the sodium carboxymethylcellulose (CMC e.g. TEXTURACEL™ 20000 PA 07) are prepared by adding the appropriate amount of the CMC powder to the appropriate amount of water in order to achieve a concentration of 1% while stirring with an overhead lab stirrer at ambient temperature for at least 1 h. The viscosity is investigated with a rheometer (e.g. Anton Paar MCR 501) with a cup and bob geometry (e.g. CC-27) at 20 degrees C. The viscosity is in the range from 10 mPa·s to 15 000 mPa·s; in some embodiment, will exhibit a viscosity of between 15 and 12 000 mPa·s, such as between 20 and 11 000 mPa·s, or between 25 and 10 000 mPa·s when so measured.

The solution for the viscosity measurements of the methylcellulose (MC e.g. Methocel™ BIND 112) are prepared by adding the appropriate amount of the MC powder to the appropriate amount of water in order to achieve a concentration of 2% while stirring with an overhead lab stirrer at ambient temperature; afterwards the solutions are cooled to temperature below 5° C. and a stirred for at least 3 h. The viscosity is investigated with a rheometer (e.g. Anton Paar MCR 501) with a cup and bob geometry (e.g. CC-27) at 5 degrees C. The viscosity is in the range from 15 mPa·s to 100 000 mPa·s; in some embodiment, will exhibit a viscosity of between 50 and 80 000 mPa·s, such as between 100 and 75 000 mPa·s, or between 150 and 70 000 mPa·s when so measured.

### EXPERIMENTAL SECTION

### Example 1

### Equipment Used to Manufacture the Formulation

1. Overhead stirrer
2. Weighing balance

### Liquid Alginate Preparation:

Composition 1:

| Sr. | | | Quantity | Quantity |
|---|---|---|---|---|
| No | Ingredients | Function | (gm) | (% w/w) |
| 1 | Sodium alginate Protanal ® LFR | Raft forming polymer | 30.0 | 3.00 |
| | 5/60 | | | |
| 2 | Sodium bicarbonate | Buoyancy agent | 26.7 | 2.67 |
| 3 | Calcium carbonate | Cross linking agent | 16.0 | 1.60 |
| 4 | Sodium carboxymethylcellulose | Viscosifier | 5.0 | 0.50 |
| | (TEXTURACEL ™ 20000 PA 07) | | | |
| 5 | Xanthan gum | Viscosifier | 1.30 | 0.13 |
| | (GRINDSTED ® Xanthan 80 PRM | | | |
| | Plus) | | | |
| 6 | Methyl (E218) | Preservative | 4.0 | 0.40 |
| | parahydroxybenzoate (or sodium | | | |
| | salt) | | | |
| 7 | Propyl (E216) | Preservative | 0.6 | 0.06 |
| | parahydroxybenzoate | | | |
| 8 | Sodium saccharin | Sweetener | 2.7 | 0.27 |
| 9 | DI water QS (In ml) | Vehicle | q.s. 1000 gm | 91.37 |

### Process:

1. Add and dissolve sodium bicarbonate, calcium carbonate, and sodium saccharin in purified water. To this, add Sodium alginate stir the mixture for 30 min.
2. Add Xanthan gum in the step 1. Stir the mixture for 30 min.
3. Add and dissolve sodium carboxymethyl cellulose into purified water under stirring.
4. Add step 3 into 2 and mix the dispersion well.
5. Add and dissolve methyl paraben and propyl paraben into hot water.
6. Add step 5 to 4 and mix well under stirring. Stir the mixture for 30 min.

### Raft Strength Evaluation:

### Raft Strength Testing Assay:

1. Add 20 ml of raft suspension into 150 ml of 0.1M HCl, maintained at 37° C. in a 250 ml low-form glass beaker having an internal diameter of 60 to 70 mm. An L-shaped stainless-steel wire probe, comprising 1 mm diameter 316 gauge stainless steel having a 90 mm vertical arm with a hook at the top and a 20 mm horizontal arm such that the vertical arm of the probe hangs down the centre axis of the beaker and the horizontal arm is in the lower third of the acid, was kept upright while adding suspension.
2. The beaker was kept at 37° C. for 30 min.
3. After 30 min of raft development, the beaker was placed on the TX Plus Texture Analyzer (Model-TX plus, make-stable microsystem, UK) using 5.0 kg load cell.
4. The wire probe was hooked onto the Texture Analyzer arm and pulled vertically up through the raft at a rate of 5 mm/s.
5. The force (g) required to pull the wire probe up through the raft was recorded by the Texture Analyzer. The variability of the raft strength is expressed as the Relative standard deviation (RSD) expressed in percentage. It is calculated by multiplying the standard deviation by 100 and dividing by the mean of the raft strength value.

### Raft Volume and Raft Weight Test:

1. Pre-weigh the beaker (W1). Add 20 ml of raft suspension into 150 ml of 0.1M HCl, maintained at 37° C. in a 250 ml low-form glass beaker.
2. The beaker was kept at 37° C. for 30 min.
3. After 30 min of raft development.
4. The position to which the top of each raft reached was marked on the outside of the beaker. Weigh the beaker (W2).
5. The raft was then removed from the beaker by carefully decanting off the sub natant liquid and tipping the raft into a pre-tared plastic weighing boat.
6. This was left to stand for 30 s, excess sub natant liquid was drained off, and the raft was weighed (W3).
7. Remaining liquid was removed from the inside of the beaker with a paper towel, and it was then refilled with water to the marked position and weighed (W4).
8. The volume of each raft was then calculated from the formula: raft volume=(W4−W1)−(W2−W1−W3), where raft volume is measured in ml. All weights are measured in g.

| | | Test product |
|---|---|---|
| | Reference Product | (with |
| | (Gaviscon) | TEXUTURECEL) |
| Raft Strength (G) (BP | 12.23 (23.9%) | 13.00 (6.47%) |
| Specification: NLT 7.5 g) | | |
| Raft Weight (g) | 53.00 (2.6%) | 46.90 (6.14%) |
| Raft Thickness (mm) | 31.76 (2.3%) | 29.10 (0.75%) |

(n = 3)

The raft strength variation data of reference product and test composition is listed below and showed improvement in the raft strength variation observation.

| | Raft Strength | Std Dev | % RSD |
|---|---|---|---|
| Test product | 13.00 | 0.8 | 6.47 |
| (with | | | |
| TEXUTURECEL) | | | |
| Gaviscon | 12.23 | 2.9 | 23.9 |

(n = 3)

Composition 2:

| Sr. | | | Quantity | Quantity |
|---|---|---|---|---|
| No | Ingredients | Function | (gm) | (% w/w) |
| 1 | Sodium alginate Protanal ® LFR | Raft forming polymer | 30.0 | 3.00 |
| | 5/60 | | | |
| 2 | Sodium bicarbonate | Buoyancy agent | 26.7 | 2.67 |
| 3 | Calcium carbonate | Cross linking agent | 16.0 | 1.60 |
| 4 | METHOCEL ™ Bind 112 | Viscosifier | 13.33 | 1.33 |
| 5 | Xanthan gum (GRINDSTED ® | Viscosifier | 2.0 | 0.2 |
| | Xanthan 80 PRM Plus) | | | |
| 6 | Methyl (E218) | Preservative | 4.0 | 0.40 |
| | parahydroxybenzoate (or | | | |
| | sodium salt) | | | |
| 7 | Propyl (E216) | Preservative | 0.6 | 0.06 |
| | parahydroxybenzoate | | | |
| 8 | Sodium saccharin | Sweetener | 2.7 | 0.27 |
| 9 | DI water QS (In ml) | Vehicle | q.s. 1000 gm | 90.47 |

### Process:

### Preparation of METHOCEL™ Bind 112 Gel:

Add METHOCEL™ Bind 112 (methylcellulose) to purified water (temperature 20-25° C.) at room temperature while stirring with an overhead lab stirrer. After complete dispersion, stir the solution for 120 min at 750 rpm in cold condition (temperature below 5° C.).

### Preparation of Raft Composition

1. Add and dissolve sodium bicarbonate, calcium carbonate, and sodium saccharin in purified water. To this, add Sodium alginate and stir the mixture for 30 min.
2. Add prepared METHOCEL™ Bind 112 gel into step 2 under stirring. Stir the mixture for 1 hour
3. Add Xanthan gum and stir the mixture for 30 min.
4. Add and dissolve methyl paraben and propyl paraben in hot water.
5. Add from step 4 to 3 and mix well under stirring. Stir the mixture for 30 min.

### Raft Strength Evaluation:

For procedure refer to procedure listed in composition 1

| | | Test product |
|---|---|---|
| | Reference Product | (with METHOCEL ™ |
| | (Gaviscon) | Bind 112) |
| Raft Strength (G) (BP | 12.23 (23.9%) | 12.07 (14.9%) |
| Specification: NLT 7.5 g) | | |
| Raft Weight (g) | 53.00 (2.6%) | 48.6 (4.5%) |
| Raft Thickness (mm) | 31.76 (2.3%) | 29.32 (1.8%) |

(n = 3)

The raft strength variation data of reference product and test composition is listed below and showed improvement in the raft strength variation observation.

| | Raft | | |
|---|---|---|---|
| | Strength | Std Dev | % RSD |
| Test product | 12.07 | 1.8 | 14.9 |
| (with METHOCEL ™ | | | |
| Bind 112) | | | |
| Gaviscon | 12.23 | 2.9 | 23.9 |

(n = 3)

1. A pharmaceutical raft-forming suspension suitable for the treatment by oral administration of gastroesophageal reflux diseases comprising:
a) an alginate in the amount of 2% (w/w) to 5% (w/w);
b) a cellulose derivative polymer selected from the group consisting of hydroxypropyl methylcellulose (HPMC), carboxymethyl cellulose (CMC), and methylcellulose (MC), hydroxypropyl cellulose (HPC); and mixtures thereof;
c) a carbonate;
d) a multivalent alginate cross-linking ion in an amount of about 1.1% (w/w) to 2.2% (w/w);
e) at least one viscosifier selected from i) a hydrocolloid, and ii) a pharmaceutically acceptable poly-acrylic acid; and
f) water or other pharmaceutical vehicle.

2. The pharmaceutical raft-forming suspension according to claim 1, wherein the alginate is a salt of alginic acid.

3. The pharmaceutical raft-forming suspension according to claim 1, where the multivalent ion is a calcium ion.

4. The pharmaceutical raft-forming suspension according to claim 1, wherein the carbonate is an alkali metal carbonate.

5. The pharmaceutical raft-forming suspension according to claim 1, which suspension further comprises a preservative.

6. The pharmaceutical raft-forming suspension according to claim 1, which suspension further comprises a flavoring and/or sweetening compound.

7. The pharmaceutical raft-forming suspension according to claim 1, wherein the alginate is present in an amount of at least 2.5% (w/w).

8. The pharmaceutical raft-forming suspension according to claim 1, wherein the alginate is present in an amount of not more than 4.0% (w/w).

9. The pharmaceutical raft-forming suspension according to claim 1, wherein the cellulose derivative polymer is present in an amount of at least 0.1% (w/w).

10. The pharmaceutical raft-forming suspension according to claim 1, wherein the cellulose derivative polymer is present in an amount of not more than 2.0% (w/w).

11.-13. (canceled)

14. The pharmaceutical raft-forming suspension according to claim 1, wherein the cellulose derivative polymer is methylcellulose.

15. The pharmaceutical raft-forming suspension according to claim 14, wherein the methylcellulose has a viscosity in the range from 15 mPa's to 100 000 mPa's as measured at 5° C. at a concentration of 2% in water at a shear rate of 10 s⁻¹.

16. The pharmaceutical raft-forming suspension according to claim 14, wherein the methylcellulose has s23/s26 from 0.10 to 0.24, wherein s23 is the molar fraction of anhydroglucose units wherein only the two hydroxy groups in the 2- and 3-positions of the anhydroglucose unit are substituted with methyl groups and wherein s26 is the molar fraction of anhydroglucose units wherein only the two hydroxy groups in the 2- and 6-positions of the anhydroglucose unit are substituted with methyl groups.

17. The pharmaceutical raft-forming suspension according to claim 1, wherein the carbonate is present in an amount of 1.5-5% (w/w).

18. The pharmaceutical raft-forming suspension according to claim 1, wherein the multivalent alginate cross-linking ion is present in an amount of 1.4-1.8% (w/w).

19. The pharmaceutical raft-forming suspension according to claim 1, wherein the viscosifier is present in an amount of 0.05% (w/w)-1.0% (w/w).

20. The pharmaceutical raft-forming suspension according to claim 1, wherein the suspension has a raft strength as measured on a Texture Analyzer as described in Raft strength Testing Assay which is higher than 7.5 g.

21. The pharmaceutical raft-forming suspension according to claim 1, wherein the suspension has a raft strength variability which is less than 20 percent relative standard deviation (% RSD).

22. A method for the preparation of a raft-forming suspension comprising the steps of mixing the components of:
a) an alginate in the amount of 2% (w/w) to 5% (w/w);
b) a cellulose derivative polymer selected from the group consisting of hydroxypropyl methylcellulose (HPMC), carboxymethyl cellulose (CMC), and methylcellulose (MC), hydroxypropyl cellulose (HPC); and mixtures thereof;
c) a carbonate;
d) a multivalent alginate cross-linking ion in an amount of about 1.1% (w/w) to 2.2% (w/w);
e) at least one viscosifier selected from i) a hydrocolloid, and ii) a pharmaceutically acceptable poly-acrylic acid; and
f) water or other pharmaceutical vehicle.

23. (canceled)

24. A method for the treatment of a gastroesophageal reflux disease comprising the administering of an effective amount to a subject in need thereof of a raft-forming suspension comprising:
a) an alginate in the amount of 2% (w/w) to 5% (w/w);
b) a cellulose derivative polymer selected from the group consisting of hydroxypropyl methylcellulose (HPMC), carboxymethyl cellulose (CMC), and methylcellulose (MC), hydroxypropyl cellulose (HPC); and mixtures thereof;
c) a carbonate;
d) a multivalent alginate cross-linking ion in an amount of about 1.1% (w/w) to 2.2% (w/w);
e) at least one viscosifier selected from i) a hydrocolloid, and ii) a pharmaceutically acceptable poly-acrylic acid; and
f) water or other pharmaceutical vehicle.

25. (canceled)
